(19) United States
(12) Patent Application Publication
(10) Pub. No.: US 2013/0190195 A1
(43) Pub. Date: Jul. 25, 2013

(54) NUCLEIC ACID DETECTION BY BRIDGE AMPLIFICATION ON CODED PARTICLES

(71) Applicant: Man Cheng, San Ramon, CA (US)

(72) Inventor: Man Cheng, San Ramon, CA (US)

(73) Assignee: Bio-Rad Laboratories, Inc., Hercules, CA (US)

(21) Appl. No.: 13/555,898

(22) Filed: Jul. 23, 2012

Related U.S. Application Data
(60) Provisional application No. 61/524,458, filed on Aug. 17, 2011.

(51) Int. Cl. C12Q 1/68

(52) U.S. Cl. CPC C12Q 1/6809
USPC 506/9; 435/6.12; 506/16

(57) ABSTRACT

Particles that generate electromagnetic spectra upon appropriate illumination and in which the spectra can be converted to codes that reflect differences in the spectra are functionalized with a plurality of identical primer pairs on which bridge amplification can be performed. Using these particles, nucleic acids in a biological sample are amplified and exposed to a label that sends a signal only when bound to double-stranded DNA. The code for each particle is correlated with the primer pairs, and the captured nucleic acids are thus detected and identified. The procedure is useful for multiplex DNA or RNA detection as well as counting of target DNA and RNA molecules in the sample.

### CROSS-REFERENCE TO RELATED APPLICATION

This application claims the benefit of U.S. Provisional Patent Application No. 61/524,458, filed Aug. 17, 2011, the contents of which are incorporated herein by reference.

### BACKGROUND AND SUMMARY OF THE INVENTION

The polymerase chain reaction (PCR), well known as a reaction that amplifies DNA by producing a multitude of copies of DNA molecules from individual starting molecules, is used in a variety of procedures including DNA sequencing, functional analyses of genes, diagnoses of hereditary diseases, the identification of genetic fingerprints, and the detection and diagnosis of infectious diseases, as well as analyses of mRNA by reverse transcriptase PCR (RT-PCR) to determine gene expression in cells and tissues, and RNAs in general for various purposes. While PCR was originally developed in the liquid phase, the development of solid-phase PCR offered a number of advantages, including a greater ease of reaction control and product manipulation, as well as the ability to perform the procedure on a multiplex basis.

The present invention incorporates the use of spectral codes into solid-phase PCR, thereby enabling individual nucleic acids as well as panels of nucleic acids to be detected, both in terms of presence or absence of particular nucleic acids and quantitative determinations of particular nucleic acids, while identifying each nucleic acid that is detected and differentiating among different nucleic acids in a single sample. The technique is applicable to all nucleic acids, including DNA and the various RNAs such as mRNA, piRNA, siRNA, and ncRNA.

The spectral codes are obtained by the use of optically readable particles, and PCR occurs on the particles each of which is functionalized with a multitude of identical pairs of primers, the first primer of each pair being hybridizable to a single target nucleic acid in a target-specific manner and the second primer being hybridizable to a nucleic acid strand that is complementary to the same target nucleic acid to which the first primer is hybridized. The procedure that utilizes these multiple primer pairs is known as “bridge amplification,” and it generates multiple copies of individual nucleic acids in the sample in double-stranded form attached to each particle. Optical reading is achieved through coded optical information on each particle, the information varying among the particles such that the particle population is classified into subpopulations by the different codes. The codes in turn are correlated with the primer pairs and hence with the target nucleic acids, such that each primer pair, and thus each target nucleic acid, is associated with an optical code that is distinguishable from all other optical codes. Once amplification and labeling are performed, detection of both the double stranded nucleic acids on the particles and of the optical information are achieved by individually known methods, and can be done without separating the subpopulations from each other. Information regarding either the presence of particular nucleic acids in the original sample, the amounts of each, or both, can thus be obtained along with identifications of the particular nucleic acids that are detected and/or quantified. This combination of target-specific primers and optically readable particles is applied in accordance with this invention to a variety of assays for nucleic acids, utilizing various protocols, as illustrated below.

### BRIEF DESCRIPTION OF THE FIGURES

FIG. 1 is a diagrammatic representation of functionalized particle subpopulations in accordance with the present invention.

FIG. 2 is a diagrammatic representation of a single functionalized particle subpopulation in accordance with the present invention being used in a procedure for detection of a specified mRNA in a sample.

FIG. 3 is a diagrammatic representation of a series of functionalized particle subpopulations in accordance with the present invention being used in a procedure for counting of three specified nucleic acids in a sample.

### DETAILED DESCRIPTION OF THE INVENTION AND SELECTED EMBODIMENTS

While the polymerase chain reaction (PCR) and its use in bridge amplification are known in the art, the following description is a review of each.

PCR is commonly performed in a reaction mixture that includes the nucleic acid to be amplified, i.e., the template nucleic acid, plus primers complementary to the 3′ ends of each of the complementary strands of the template, a DNA polymerase, deoxynucleoside triphosphates (dNTPs), a buffer, and appropriate cations, typically potassium and either magnesium or manganese. Following an initial heating step that is generally performed at about 96° C. to about 98° C. for one to nine minutes to activate the polymerase, the reaction mixture is subjected to a series of repeats, or “cycles,” of a temperature change sequence. Each cycle includes (a) a short continuation of the initial heating step, at the same temperature and typically for about ten seconds to about thirty seconds, and (b) an annealing and elongation step (annealing the primers and, once annealed, elongating them by reaction with the deoxynucleoside triphosphates to add nucleotides), at a temperature in the range of about 60° C. to about 72° C. for a period of about one minute for each thousand bases in the template. Each cycle replicates a single-strand DNA.

In bridge amplification, as explained for example in Boles et al., U.S. Pat. No. 6,300,070 B1, “Solid Phase Methods for Amplifying Multiple Nucleic Acids,” Oct. 9, 2001, the primer that is complementary to the 3′ end of the template nucleic acid is the first primer of each pair that is covalently attached to the solid particle. When a sample containing the template nucleic acid is contacted with the particle and a single thermal cycle is performed, the template molecule is annealed to the first primer and the first primer is elongated in the forward direction by addition of nucleotides to form a duplex molecule consisting of the template molecule and a newly formed DNA strand that is complementary to the template. In the heating step of the next cycle, the duplex molecule is denatured, releasing the template molecule from the particle and leaving the complementary DNA strand attached to the particle through the first primer. In the annealing stage of the annealing and elongation step that follows, the complementary strand hybridizes to the second primer, which is complementary to a segment of the complementary strand at a location removed from the first primer. This hybridization causes the complementary strand to form a bridge between the first and second primers secured to the first primer by a covalent bond and to the second primer by hybridization. In the elongation stage, the second primer is elongated in the reverse direction by the addition of nucleotides in the same reaction mixture, thereby converting the bridge to a double-stranded bridge. The next cycle then begins, and the double-stranded bridge is denatured to yield two single-stranded nucleic acid molecules, each having one end attached to the particle surface via the first and second primers, respectively, with the other end of each unattached. In the annealing and elongation step of this second cycle, each strand hybridizes to a further complementary primer, previously unused, on the same particle, to form new single-strand bridges. The two previously unused primers that are now hybridized elongate to convert the two new bridges to double-strand bridges.

Each successive cycle doubles the number of bridges formed until any desired number of double-stranded bridges that are generated from the single template molecule. The number of cycles can vary; a number within the range of 5 to 50 cycles is an example. In the practice of this invention, the double-stranded bridges once formed are labeled by contact with a label that either binds only to double-stranded nucleic acids or that is significantly more detectable when bound to double-stranded nucleic acids than when bound to single-strand nucleic acids. Examples of such labels are fluorescent tags such as SYBR Green® I (Invitrogen, Inc., Carlsbad, Calif., USA), EvaGreen® (Biotium, Inc., Hayward, Calif., USA), SYTO® 9 (Invitrogen, Inc.), LCGreen® (Idaho Technology, Inc., Salt Lake City, Utah), and YO-PRO®-1 (Invitrogen, Inc.). The labels can either be included among the initial reaction materials or added to the reaction mixture after all cycles are complete.

Since at least one of the primers of each pair on a given particle is specific for a particular template, that template serves as a target nucleic acid, and the bridge formed by any given primer pair will be one that includes only the target nucleic acid for the target-specific primer(s). Since all primer pairs on each particle are identical, the molecules of only one target nucleic acid will form bridges on any single particle. The term “target-specific” as used herein thus means that the primer has a sufficient number of bases to distinguish it from primers that are target-specific for other target nucleic acids, i.e., the base sequence of the primer is complementary only to a single target nucleic acid among any other nucleic acids that may be present in the sample. The number of bases that are needed to establish target specificity can vary widely, depending on the number of different nucleic acids in the sample and their diversity within the sample. In many cases, the segment of the primer that provides the target specificity will be from about 10 to about 500 bases, or in some cases from about 30 to about 300 bases.

While the primers attached to a single particle will form double-strand nucleic acid bridges from a target nucleic acid of only one sequence, the amplification can begin from a single copy of that target nucleic acid from a sample or from two or more copies. In either case, the number of bridges formed will be limited by the number or primer pairs on the particle. To allow bridges to form, the spacing between individual primers in a pair will be less than the length of the target nucleic acids, and yet great enough that each individual strand formed on the particle will preferably form a bridge between a pair of primers that are closest to each other. These spacings can be controlled by control of the number of primer pairs attached to any single particle. Furthermore, while a single particle can have target specificity (through its primers) for a single target molecule, multiple particles with target specificity for the same target molecule can be used. In certain embodiments of this invention, as seen below, multiple particles with the same target specificity allow for quantitative analyses of individual target nucleic acids. A still further variable among embodiments of this invention is the number of target molecules to be identified in a given procedure. In certain embodiments, all particles will have identical target specificity and thereby permit detection or quantification of a single target nucleic acid. In other embodiments, particles that are grouped or classified into subpopulations are used, each subpopulation containing as few as one particle or two or more particles, possibly thousands, and each subpopulation being target-specific for a different target nucleic acid, for performing multiplex detections. There is no limit to the number of subpopulations, although in certain cases, the number may be limited by the means by which the subpopulations are differentiated in the label detection stage.

The spectral codes are derived from electromagnetic spectra that are generated by the solid particles upon illumination. The term “codable” in connection with the spectra is used herein to denote that each spectrum can be converted to a code, including machine-readable codes, that reflects certain selected differences between the spectra of different particles.

Examples of electromagnetic spectra are fluorescence spectra, light scattering spectra, absorbance spectra, and reflectance spectra. Further examples will be apparent to those of skill in the art. Fluorescence spectra can be generated by incorporating various fluorescent materials in the particles to produce spectra that differ due to the different fluorescent materials or different proportions or concentrations of the materials. Spectra can also be generated by light scattering, light emission, or combinations of light scattering and light emission. Side angle light scatter varies with particle size, granularity, absorbance and surface roughness, while forward angle light scatter is mainly affected by size and refractive index. Thus, varying any of these qualities can serve as a means of generating spectra. Light emission can be varied by incorporating fluorescent materials in the particles and using fluorescent materials that have different fluorescence intensities or that emit fluorescence at different wavelengths, or by varying the amount of fluorescent material incorporated. By using a plurality of fluorescent emissions at various wavelengths, the spectra can based on wavelength differences. Absorbance spectra can be obtained by applying light to the particles and detecting the strength of the laterally (side-angle) scattered light while the strength of the forward-scattered light is relatively unaffected. The difference in absorbance between various colored dyes associated with the particles is determined by observing differences in the strength of the laterally scattered light.

The spectral codes are derived from electromagnetic spectra that are generated by the solid particles upon illumination. The term “codable” as used herein in describing the spectra denotes that each spectrum can be converted to a code, including machine-readable codes, that reflects certain selected differences between the spectra of different particles. The codes can for example be binary or alphanumeric. When the spectra contain an array of peaks, the codes can represent the locations of the peaks in the spectra (i.e., the wavelengths at which the peaks occur), the intensities of the peaks, or both the locations and intensities.

Optically-readable particles that generate reflectivity spectra are described by Meade, S. O., et al. “Multiplexed DNA Detection Using Spectrally Encoded Porous SiO₂ Photonic Crystal Particles,” Anal. Chem. 2009, 81, 2618-2625, and by Sailor, M. J., et al., in United States Patent Application Publications No. US 2005/0042764 A1, published Feb. 24, 2005, No. US 2007/0051815 A1, published Mar. 8, 2007, and No. US 2007/0148695 A1, published Jun. 28, 2007. As explained in these references, each optically readable particle is solid and chemically inert other than the primers with which the particle is functionalized, and each particle has a stratified refractive index gradient that provides the particle with a reflectivity spectrum that typically includes an interference pattern. Different particles, or different subpopulations of particles, have different refractive index gradients and thus different spectra. Each spectrum can be divided into segments with each segment containing at most a single peak. Each segment can then be assigned a binary code indicating presence or absence of a peak in that segment, presence being defined as a spectral intensity that exceeds a threshold value, and absence as a spectral intensity that remains below the threshold. The various combinations of segments in which peaks are either present or absent produce a series of binary codes. By increasing the number of refractive index strata in each particle and correspondingly the number of segments to be included in each spectrum, the number of distinguishable codes to be generated can be as large as desired.

Materials from which the particles are made, means of achieving a refractive index gradient in a single particle and of varying the gradients among different particles or groups of particles are now known in the art per the Meade et al. and Sailor et al. references cited above, as are the means of reading the spectral codes. The particles can be manufactured from any of various forms of silica, for example, and refractive index strata can be formed from porosity strata by anodic etching with an etching waveform of multiple superimposed sine waves. Reflectivity occurs at the interfaces of the strata, and the spectra can be generated by either UV light, visible light, near-infrared light, or infrared light, depending on the reflectivities. The optimal particle shape is one that lends itself to optimal reading the reflectivity spectra, and some of the best shapes for this purpose are flat wafers, including disks. Individual wafers or disks can be of microscopic dimensions, such as for example disks that are 10-100 microns in diameter.

The primers can be attached to the particles by covalent bonds, using conventional coupling chemistry. A covalent linkage between 5′-amino-modified primers and epoxy silane-derivatized glass, for example, can be achieved by the use of an epoxide opening reaction, as described by Beattie, K. L., et al., “Degenerate Oligonucleotide Primed-Polymerase Chain Reaction and Capillary Electrophoretic Analysis of Human DNA on Microchip-Based Devices,” Clin. Chem. 41, 700-706 (1995). Alternatively, primers that are 5′-succinylated can be coupled to amino-derivatized glass, as described by Joos, B., et al., “Covalent attachment of hybridizable oligonucleotides to glass supports,” Anal. Biochem. 247, 96-101 (1997). Primers that are 5′-sulfide modified can be coupled to thiol-derivatized glass through disulfide bonds, as described by Rogers, Y. H., et al., “Immobilization of oligonucleotides onto a glass support via disulfide bonds: A method for preparation of DNA microarrays,” Anal. Biochem. 266, 23-30 (1999). Attachment can also be achieved through crosslinkers such as phenyldiisothiocyanateor maleic anhydride, again using known chemistries. Still other attachment chemistries will be apparent to those skilled in the art.

Once bridge amplification has been performed and a multitude of double-stranded bridges are formed on the particles and labeled, both detection of the labels and generation and decoding of reflectivity spectra are performed. As noted above, label detection provides an indication of the presence of the target nucleic acid(s) in the sample, and the codes derived from the reflectivity spectra provide a means of identifying the target nucleic acids that initiated the bridge formation. Generating and decoding of the reflectivity spectra is accomplished by means known in the art and described in the literature cited above. A laser, for example, of appropriate wavelength for optimal reflection in conjunction with a scanning monochromatic camera can be used to generate and capture the spectra, while readily available software, examples of which are MALTAB and IMAX, can be used to convert the spectra to binary codes.

Nucleic acids that can be targeted, detected, and quantified in the practice of this invention include both DNA and RNA. One particularly useful application of this invention is the detection and quantification of mRNA, although other applications will be readily recognizable as logical extensions. Samples on which the analyses can be performed include biological samples in general, examples of which are plasma, serum, urine, and cerebrospinal fluid.

The following protocols are offered for purposes of illustration and are not intended to define or limit the scope of the invention in any manner.

### Example 1

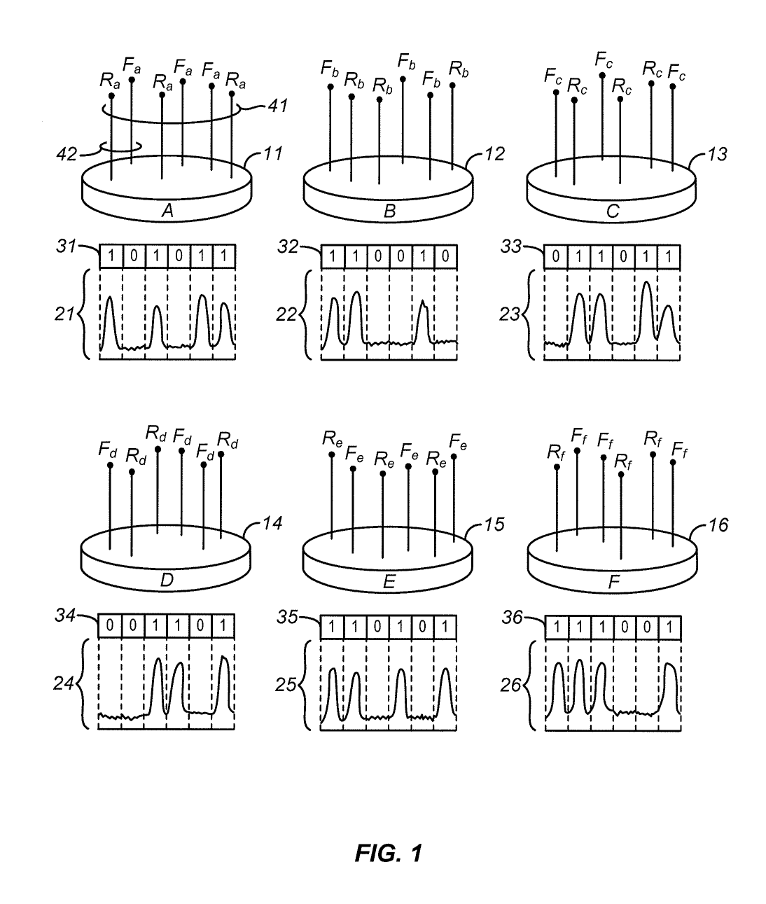

This example illustrates a population of functionalized particles useful in an application of this invention to a multiplex detection of nucleic acids. The population in this example consists of six subpopulations, each subpopulation functionalized with a different set of primer pairs. The functionalized particles are thus useful in detecting the presence or absence of six distinct nucleic acids simultaneously in a single sample. The subpopulations are illustrated in a representative manner in FIG. 1.

The particles in this example are microdisks, and each subpopulation is represented by a single disk 11, 12, 13, 14, 15, respectively, designated by the letters A, B, C, D, E, F, although each subpopulation in an actual implementation will contain many identical disks. The six disks have distinct reflectivity spectra 21, 22, 23, 24, 25, 26, respectively, which translate to six distinct binary photonic codes 31, 32, 33, 34, 35, 36, for the six disks 11, 12, 13, 14, 15, respectively. Each disk also has three identical pairs of primers 41, each pair 42 including a forward primer F and a reverse primer R. Here again, in an actual implementation of this concept, the number of primer pairs on each disk will be considerably greater than three. Each distinct primer pair is target-specific to a distinct nucleic acid, with the target specificity of primers on the A disks designated by the subscript a, the target specificity of primers on the B disk designated by the subscript b, the target specificity of primers on the C disk designated by the subscript c, the target specificity of primers on the D disks designated by the subscript d, the target specificity of primers on the E disks designated by the subscript e, and the target specificity of primers on the F disks designated by the subscript f.

### Example 2

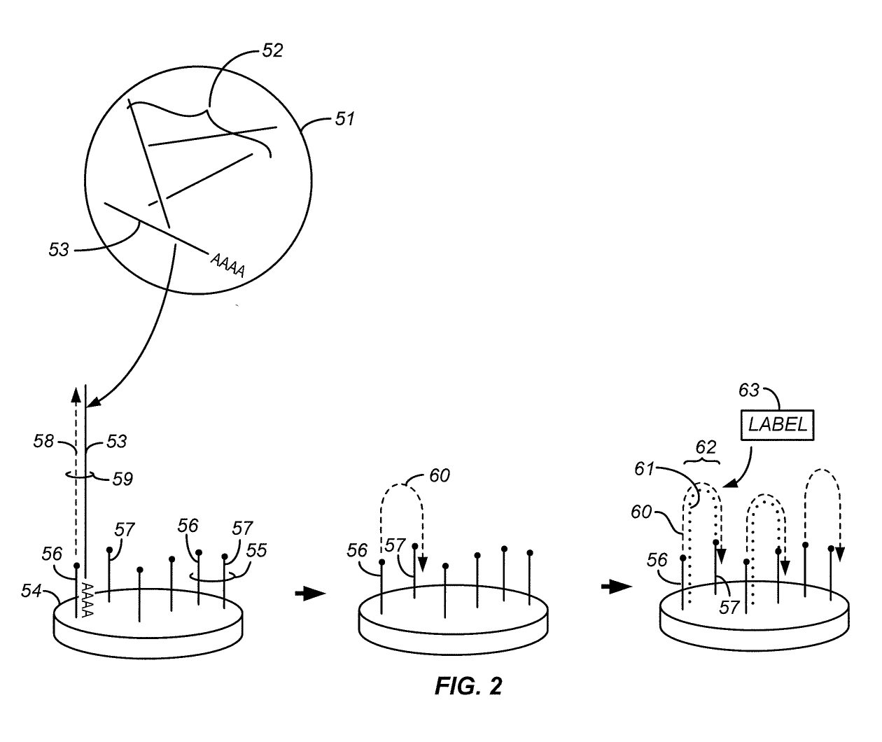

This example illustrates a bridge amplification procedure performed on a single particle in accordance with the present invention, to detect the presence of a single designated mRNA in a sample. This assay is illustrated in FIG. 2.

The sample 51 in this example contains several mRNAs 52, one of which 53 is the mRNA whose presence is sought to be determined, i.e., the target mRNA. The sample is contacted with an optically coded microdisk 54, or a population of identical such disks represented by the single disk shown, the disk having attached to its surface a number of pairs 55 of primers, each pair consisting of a reverse primer 56 and a forward primer 57. The contact results in the target mRNA 53 hybridizing to the reverse primer 56 of one primer pair, since that primer is target-specific for the target mRNA only. Thus captured by the reverse primer 56, the target mRNA is subjected to reverse transcription to produce a cDNA 58 that is complementary to the target mRNA, the two forming a double-stranded nucleic acid 59. The captured mRNA 53 is then degraded by RNase H, and the single-strand cDNA 58 that remains attached to the disk hybridizes to a neighboring forward primer 57 to form a single-strand cDNA bridge 60 between the reverse and forward primers. Bridge PCR is then performed to first elongate the forward primer 57 along the cDNA 60 to form a complementary strand 61 and the two strands form a double-stranded bridge 62 between each pair of primers. A label 63 such as any of those listed above then attaches to each of the double-stranded bridges, and both the label and the photonic code of the disk are read to indicate the presence of the mRNA in the sample.

An analogous procedure can be followed for the detection of a single designated DNA.

### Example 3

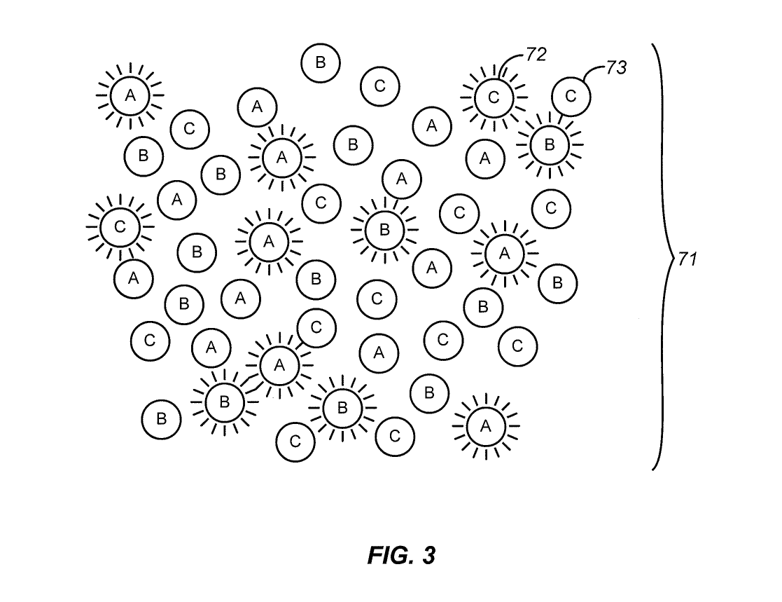

This example illustrates a bridge amplification procedure to obtain quantitative counts of individual mRNAs in a sample. This procedure is illustrated by FIG. 3.

The procedure of Example 2 is followed, except that disk population 71 consisting of a number of disk subpopulations are used rather than one, each subpopulation having its own photonic code distinguishable from the others, and each subpopulation consisting of a number of disks that exceeds the range of the suspected number of mRNA molecules which each subpopulation will amplify. Different subpopulations are designated by different letters, using A, B, and C. The excess of disks in each subpopulation is enough to assure that no more than one mRNA molecule will bind to, and be amplified on, each disk. As a result of the procedure, only those disks that captured an mRNA molecule emit a fluorescent signal, and both the number of disks emitting the fluorescent signal and the photonic code of each disk are read. With this information, the number of molecules of each target mRNA is determined, without separating the subpopulations. FIG. 3 shows the differentiation between disks emitting fluorescent signals 72 and disks that do not 73, by showing signal rays emanating from the signal-emitting disks 72.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A method for analyzing a sample of biological fluid to detect the presence of a target nucleic acid, said method comprising:
(a) contacting said sample with a solid particle that generates a codable electromagnetic spectrum upon illumination, said particle functionalized with a plurality of identical pairs of primers, a first primer of each said pair being target-specific for said target nucleic acid, and a second primer of each said pair being hybridizable to a nucleic acid strand that is complementary to said target nucleic acid, to cause a molecule of said target nucleic acid when present in said sample to hybridize to one of said first primers, said electromagnetic spectrum being such that a code generated therefrom can be correlated with said first primer according to a predetermined correlation;
(b) exposing said particle to conditions tending to cause polymerase chain reaction and bridge amplification to occur on said particle, thereby forming a plurality of double-stranded nucleic acids on said particle, one strand of each of said double-stranded nucleic acids being a copy of said target nucleic acid when said target nucleic acid is present in said sample;
(c) contacting said particle with a fluorescent label whose fluorescence is detectably increased when said label binds to double-stranded nucleic acid, and detecting any increase fluorescence emitted by said label as an indication of the presence of said target nucleic acid in said sample; and
(d) illuminating said particle to generate said electromagnetic spectrum, detecting said spectrum so generated, converting said spectrum to said code, and correlating said code with said first primer, thereby associating any increase in fluorescence detected in (c) with said target nucleic acid.

2. A method for analyzing a sample of biological fluid to detect the presence of one or more of a panel of target nucleic acids, said method comprising:
(a) contacting said sample with a population of solid particles, each of said particles generating a codable electromagnetic spectrum upon illumination, said spectra classifying said particles into subpopulations that are distinguishable from each other by said spectra, each of said particles functionalized with a plurality of identical pairs of primers, a first primer of each said pair being target-specific for one target nucleic acid of said panel, and a second primer of each said pair being hybridizable to a nucleic acid strand that is complementary to said one target nucleic acid, to cause molecules of all of said target nucleic acids of said panel that are present in said sample to hybridize to said first primers to which said target nucleic acids are hybridizable, all particles in each subpopulation functionalized with a common pair of primers distinct from all other said subpopulations and each subpopulation having a spectrum such that a code generated therefrom can be correlated with primers that are target-specific for one target nucleic acid of said panel distinct from all other target nucleic acids of said panel;
(b) exposing said population of particles to conditions tending to cause polymerase chain reaction and bridge amplification to occur on said particles, thereby forming a plurality of double-stranded nucleic acids, one strand of each of said double-stranded nucleic acids being a copy of a target nucleic acid of said panel when said target nucleic acid is present in said sample;
(c) contacting said population of particles with a fluorescent label whose fluorescence is detectably increased when said label binds to double-stranded nucleic acid, and detecting any increase fluorescence emitted by said label as an indication of the presence of a target nucleic acid of said panel in said sample; and
(d) illuminating said particles to generate said electromagnetic spectra of each of said particles for which said increase in fluorescence is detected, detecting said spectra so generated, converting said spectra to codes, and correlating said codes with said first primers, thereby associating any increase in fluorescence so detected with a specific nucleic acid of said panel.

3. The method of claim 1 wherein said particle has a stratified refractive index gradient and said codable electromagnetic spectrum is a reflectivity spectrum generated when said particle is illuminated with a wavelength-scanning light beam, and said code is a binary code representing locations of peaks in said spectrum.

4. The method of claim 2 wherein said particles have stratified refractive index gradients and said codable electromagnetic spectra are reflectivity spectra generated when said particles are illuminated with a wavelength-scanning light beam, and said codes are binary codes representing locations of peaks in said spectra.

5. The method of claim 1 wherein said particle is dyed with a plurality of fluorophores at selected intensities and said codable electromagnetic spectrum is an emission spectrum generated when said particle is illuminated with a fluorescence-activating light beam, and said code represents locations of peaks in said spectrum, intensities of said peaks, or both locations and intensities of said peaks.

6. The method of claim 2 wherein said particles are dyed with a plurality of fluorophores at selected intensities, said codable electromagnetic spectra are emission spectra generated when said particles are illuminated with a fluorescence-activating light beam, and said codes represent locations of peaks in said spectra, intensities of said peaks, or both locations and intensities of said peaks.

7. The method of claim 2 wherein said sample is known to contain no more than a predetermined maximum number of molecules of each of said target nucleic acids of said panel, and each said subpopulation consists of particles that exceed in number said predetermined maximum, such that all molecules in said sample of each of said target nucleic acids of said panel bind to particles in step (a) by hybridization to said first primers, said method further comprising quantitatively determining the amount of each of said target nucleic acids of said panel.

8. A population of solid particles, each of said particles generating a codable electromagnetic spectrum upon illumination, said spectra classifying said particles into subpopulations that are distinguishable from each other by said spectra, each of said particles functionalized with a plurality of identical pairs of primers, a first primer of each said pair being target-specific for a distinct target nucleic acid, and a second primer of each said pair being hybridizable to a nucleic acid strand that is complementary to said target nucleic acid, all particles in each subpopulation functionalized with a common pair of primers distinct from all other said subpopulations and each subpopulation generating a spectrum such that a code representing said spectrum can be correlated with primers that are target-specific for one nucleic acid of said panel distinct from all other nucleic acids of said panel.

9. The population of claim 8 wherein said particles have stratified refractive index gradients and said codable electromagnetic spectra are reflectivity spectra generated when said particles are illuminated with a wavelength-scanning light beam, and said codes are binary codes representing locations of peaks in said spectra.

10. The population of claim 8 wherein said particles are dyed with a plurality of fluorophores at selected intensities, said codable electromagnetic spectra are emission spectra generated when said particles are illuminated with a fluorescence-activating light beam, and said codes represent locations of peaks in said spectra, intensities of said peaks, or both locations and intensities of said peaks.
